(19) United States
(12) Patent Application Publication
(10) Pub. No.: US 2025/0308517 A1
(43) Pub. Date: Oct. 2, 2025

(54) BIOLOGICAL INFORMATION PROCESSING SYSTEM, INFORMATION PROCESSING APPARATUS, BIOLOGICAL INFORMATION PROCESSING METHOD, AND NON- TRANSITORY RECORDING MEDIUM

(71) Applicants: NEC Corporation, Tokyo (JP); NATIONAL CENTER FOR GLOBAL HEALTH AND MEDICINE, Tokyo (JP)

(72) Inventors: Takafumi YOSHINAGA, Tokyo (JP); Yasushi OKADA, Tokyo (JP); Takumi TANAKA, Tokyo (JP); Shigeki KANO, Tokyo (JP); Shohei YAMAGUCHI, Tokyo (JP); Kengo MIYO, Tokyo (JP); Ryota NISHI, Tokyo (JP)

(73) Assignees: NEC Corporation, Tokyo (JP); NATIONAL CENTER FOR GLOBAL HEALTH AND MEDICINE, Tokyo (JP)

(21) Appl. No.: 19/092,084

(22) Filed: Mar. 27, 2025

(30) Foreign Application Priority Data
Mar. 28, 2024 (JP) 2024-052728

(51) Int. Cl. G10L 15/183; G06F 3/01; G06T 7/73; G10L 25/66; G16H 10/60

(52) U.S. Cl. CPC G10L 15/183; G06F 3/013; G06T 7/73; G10L 25/66; G16H 10/60; G06T 2207/30201

(57) ABSTRACT

A biological information processing system according to the present disclosure includes a wearable terminal worn by a medical worker and configured to acquire a voice, and an information processing apparatus that extracts and stores biological information of a patient from the voice acquired by the wearable terminal. The wearable terminal includes a sound collection unit that collects sound, a voice acquisition unit that acquires the collected sound, and a voice information transmission unit that transmits voice information indicating the acquired sound to the information processing apparatus. An information processing apparatus includes a voice information analysis unit that analyzes transmitted voice information and outputs character information indicating a word included in the voice information, a biological information extraction unit that extracts biological information of a patient from the output character information, and a patient information storage unit that stores the extracted biological information of the patient in a storage device.

### INCORPORATION BY REFERENCE

This application is based upon and claims the benefit of priority from Japanese patent application No. 2024-52728, filed on Mar. 28, 2024, the disclosure of which is incorporated herein in its entirety by reference.

### TECHNICAL FIELD

The present disclosure relates to a biological information processing system, an information processing apparatus, a biological information processing method, and a biological information processing program for recording biological information of a patient.

### BACKGROUND ART

Conventionally, a medical worker such as a nurse in a medical site measures biological information such as body temperature, blood pressure, pulse, and blood oxygen concentration of a plurality of hospitalized patients, and inputs the measurement result to an electronic medical record afterwards. Recently, a medical worker records a measurement result of biological information using a portable terminal. However, in such a case, the medical worker has to operate the screen using a hand, a touch pen, or the like to record the measurement results of the biological information of the plurality of hospitalized patients, which is a burden on the medical worker.

In this regard, Published Japanese Translation of PCT International Publication for Patent Application, No. 2023-545578 discloses a method of presenting, on a display device at a medical care setting, a suggested consultation action based on patient data related to a patient during a patient consultation, presenting a suggested consultation action, a patient consultation, and a portion of patient data contextually related to the patient during the patient consultation, and updating the portion of patient data presented on the display device based on measurement values generated from the suggested consultation actions.

### SUMMARY

However, since the method disclosed in Published Japanese Translation of PCT International Publication for Patent Application, No. 2023-545578 presents various information of a patient using a stationary display, it is not suitable for a situation in which a medical worker moves to a place where the patient is and records biological information of the patient.

In view of the above-described problems, an example object of the present disclosure is to provide a biological information processing system, an information processing apparatus, a biological information processing method, and a biological information processing program capable of improving convenience in recording a measurement result of biological information of a patient.

A biological information processing system according to a first example aspect of the present disclosure includes:
a wearable terminal worn by a medical worker and configured to acquire a voice; and
an information processing apparatus configured to extract and store biological information of a patient from the voice acquired by the wearable terminal, in which
the wearable terminal includes:
   a sound collection unit configured to collect the voice;
   a voice acquisition unit configured to acquire the voice collected by the sound collection unit; and
   a voice information transmission unit configured to transmit voice information indicating the voice acquired by the voice acquisition unit to the information processing apparatus, and
the information processing apparatus includes:
   a voice information analysis unit configured to analyze the voice information transmitted by the voice information transmission unit and output character information indicating a word included in the voice information;
   a biological information extraction unit configured to extract biological information of the patient from the character information output by the voice information analysis unit; and
   a patient information storage unit configured to store the biological information of the patient extracted by the biological information extraction unit in a storage device.

An information processing apparatus according to a second example aspect of the present disclosure includes:
a voice information analysis unit configured to analyze voice information indicating a voice acquired by a wearable terminal worn by a medical worker and output character information indicating a word included in the voice information;
a biological information extraction unit configured to extract biological information of a patient from the character information output by the voice information analysis unit; and
a patient information storage unit configured to store the biological information of the patient extracted by the biological information extraction unit in a storage device.

A biological information processing method according to a third example aspect of the present disclosure, causes a computer to:
analyze voice information indicating a voice acquired by a wearable terminal worn by a medical worker and output character information indicating a word included in the voice information;
extract the biological information of the patient from the output character information; and
store the extracted biological information of the patient in a storage device.

A biological information processing program according to a fourth example aspect of the present disclosure, causes a computer to:
analyze voice information indicating a voice acquired by a wearable terminal worn by a medical worker and output character information indicating a word included in the voice information;
extract the biological information of the patient from the output character information; and
store the extracted biological information of the patient in a storage device.

According to the present disclosure, it is possible to provide a biological information processing system, an information processing apparatus, a biological information processing method, and a biological information processing program capable of enhancing convenience in recording a measurement result of biological information of a patient.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a diagram illustrating an example of a biological information processing system according to the present disclosure;

FIG. 2 is a diagram illustrating an example of a configuration of a wearable terminal according to the present disclosure;

FIG. 3 is a diagram illustrating an example of a configuration of an information processing apparatus according to the present disclosure;

FIG. 4 is a diagram illustrating an example of a patient information table according to the present disclosure;

FIG. 5 is a flowchart illustrating an example of processing executed in the biological information processing system according to the present disclosure;

FIG. 6 is a flowchart illustrating another example of the processing executed in the biological information processing system according to the present disclosure;

FIG. 7 is a flowchart illustrating another example of the processing executed in the biological information processing system according to the present disclosure;

FIG. 8 is a diagram illustrating an example of an image according to the present disclosure;

FIG. 9 is a diagram for explaining an instruction to start recording of biological information with a fingertip;

FIG. 10 is a diagram illustrating another example of an image according to the present disclosure;

FIG. 11 is a diagram illustrating another example of the image according to the present disclosure;

FIG. 12 is a diagram illustrating another example of the image according to the present disclosure; and

FIG. 13 is a diagram illustrating main components included in the biological information processing system according to the present disclosure.

### EXAMPLE EMBODIMENTS

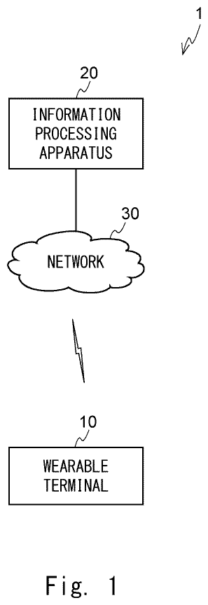

Hereinafter, an example embodiment will be described with reference to the drawings. FIG. 1 is a diagram illustrating an example of a biological information processing system 1 according to the present disclosure. The biological information processing system 1 includes a wearable terminal 10 and an information processing apparatus 20. Note that, although FIG. 1 illustrates a single wearable terminal 10, the biological information processing system 1 may include a plurality of wearable terminals 10.

The wearable terminal 10 and the information processing apparatus 20 communicate data with each other via a network 30. The network 30 may include a narrow area network such as a local area network (LAN) or the like and/or a wide area network such as the Internet.

The wearable terminal 10 is a terminal device worn by a medical worker and capable of acquiring a voice of the medical worker. Specific examples of the wearable terminal 10 include eyeglass-type wearable terminals such as smart glasses, and watch-type wearable terminals. The wearable terminal 10 can transmit and receive data by wireless communication. Details of the configuration of the wearable terminal 10 will be described later.

The information processing apparatus 20 is an information processing apparatus that extracts and stores the biological information of the patient from the voice acquired by the wearable terminal 10. Specific examples of the information processing apparatus 20 include an information processing apparatus such as a server. Details of the configuration of the information processing apparatus 20 will be described later.

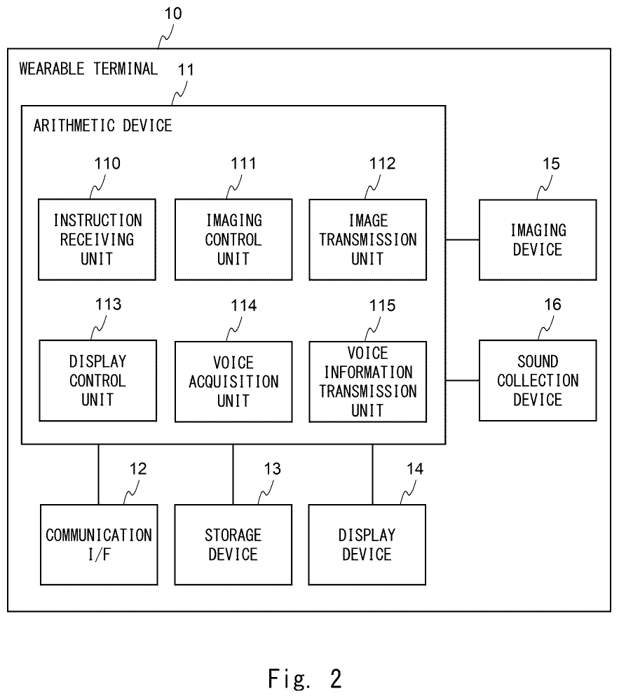

FIG. 2 is a schematic diagram illustrating a configuration of the wearable terminal 10 according to the present disclosure. The wearable terminal 10 includes an arithmetic device 11, a communication interface (I/F) 12, a storage device 13, a display device 14, an imaging device 15, and a sound collection device 16.

The arithmetic device 11 is a device that performs overall control of the wearable terminal 10. Specific examples of the arithmetic device 11 include a processor such as a central processing unit (CPU) or a micro processing unit (MPU). The arithmetic device 11 executes a program stored in the storage device 13 to execute a method defined by the program. In another example embodiment, the function executed by the arithmetic device 11 may also be executed by an integrated circuit such as a field-programmable gate array (FPGA) or an application specific integrated circuit (ASIC). These devices correspond to a computer.

The communication interface 12 is an interface that performs data communication between the wearable terminal 10 and another device.

The storage device 13 is a storage device that stores various data such as a program executed by the arithmetic device 11 and data processed by the arithmetic device 11.

The display device 14 is a device that displays various images. Various pieces of information of the patient are displayed on the display device 14. In a case where a spectacle-type wearable terminal is adopted as the wearable terminal 10, the display device 14 is a transmissive type and is disposed in front of the eyes of the wearer of the wearable terminal 10. The display device 14 corresponds to a display unit.

The imaging device 15 is a device that performs imaging under the control of the arithmetic device 11. The wearable terminal 10 may include a plurality of imaging devices 15. One of the imaging devices 15 is installed so as to be able to image the surroundings of the wearable terminal 10. In a case where the spectacle-type wearable terminal is adopted as the wearable terminal 10, another imaging device 15 is disposed so as to be able to image the field of view of the wearer via the transmission type display device 14. Furthermore, the other imaging device 15 can be disposed so as to be able to image the eyes of the wearer of the wearable terminal 10.

The sound collection device 16 is a device for collecting the voice of the wearer of the wearable terminal 10. Specific examples of the sound collection device 16 include a device such as a microphone. The sound collection device 16 corresponds to a sound collection unit.

The arithmetic device 11 executes an instruction receiving unit 110, an imaging control unit 111, an image transmission unit 112, a display control unit 113, a voice acquisition unit 114, and a voice information transmission unit 115. These functional units can be realized by a program.

The instruction receiving unit 110 is a program that receives various instructions from the wearer. The instruction received by the instruction receiving unit 110 includes an imaging instruction, a patient biological information recording start instruction, and a patient biological information recording end instruction.

The imaging instruction instructs imaging by the imaging device 15 of the wearable terminal 10. This instruction can be performed by the wearer using an input device such as a button included in the wearable terminal 10.

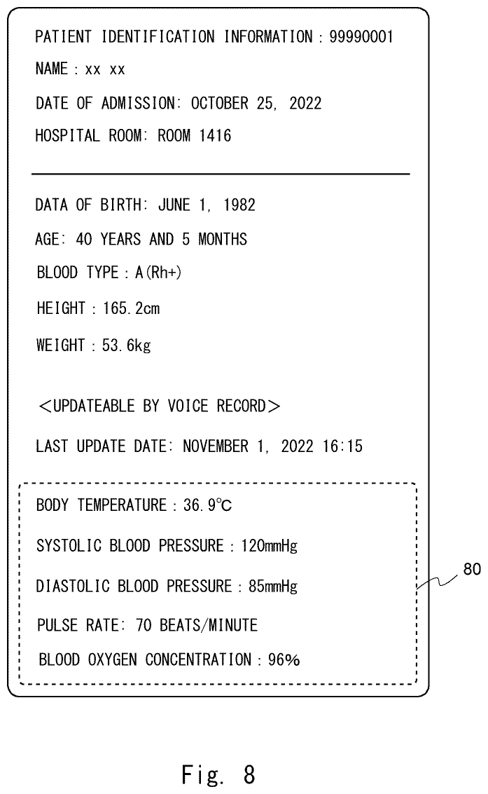

The recording start instruction of the biological information of the patient is for instructing to start recording of the biological information of the patient. The recording start instruction of the biological information of the patient is performed by a predetermined motion and voice of the wearer. For example, the recording start instruction of the biological information of the patient can be performed using the fingertip of the wearer of the wearable terminal 10. In a case where a glasses-type wearable terminal is adopted as the wearable terminal 10, the instruction receiving unit 110 determines whether the position of the fingertip of the wearer in the captured image generated by the imaging device 15 is in a predetermined area of the captured image corresponding to a predetermined area 80 of the image displayed on the display device 14 as illustrated in FIG. 8. The instruction receiving unit 110 can specify the fingertip of the wearer in the captured image using a trained model, pattern matching, or the like capable of recognizing the fingertip of the person.

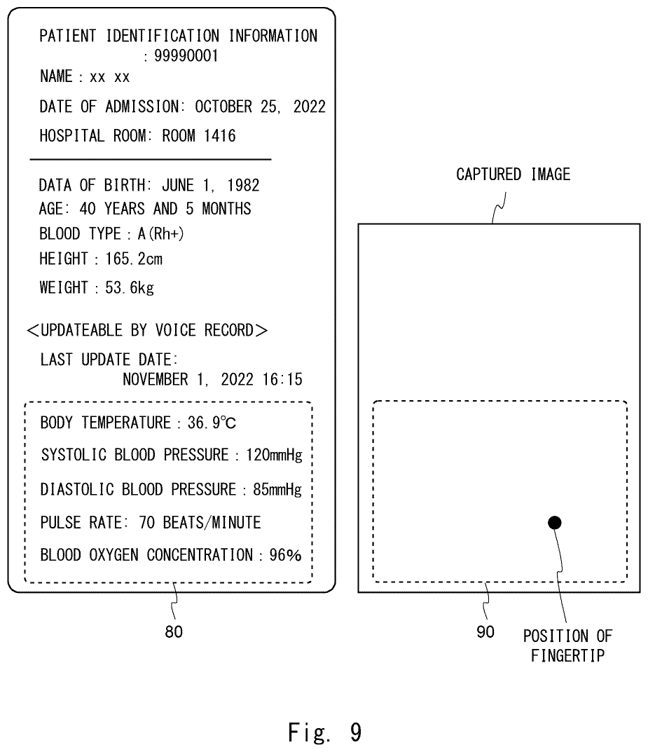

Specifically, as illustrated in FIG. 9, the instruction receiving unit 110 determines whether the position of the fingertip of the wearer in the captured image generated by imaging device 15 is in a predetermined area 90 of the captured image corresponding to the predetermined area 80 of the image displayed on the display device 14. Then, in a case where it is determined that the position of the fingertip of the wearer is in the predetermined area 90 of the captured image, the instruction receiving unit 110 receives an instruction to start the recording of the biological information of the patient.

In addition, the instruction receiving unit 110 determines whether the position of the center point of the iris of the wearer in the captured image generated by the imaging device 15 is in a predetermined area of the captured image corresponding to the predetermined area of the image displayed on the display device 14. The instruction receiving unit 110 can specify the iris of the wearer in the captured image using a trained model capable of recognizing the iris of the person, pattern matching, or the like. Then, in a case where it is determined that the position of the center point of the iris of the wearer is in the predetermined area of the captured image, an instruction to start the recording of the biological information of the patient is received.

The recording end instruction of the biological information of the patient is for instructing to end recording of the biological information of the patient. The recording end instruction of the biological information of the patient is issued by the wearer's voice.

The imaging control unit 111 is a program that controls the imaging device 15 to generate the captured image. For example, the imaging control unit 111 causes the imaging device 15 to image a code in which identification information of a patient is embedded, and generates a captured image (hereinafter, it is referred to as a “code image”.) including the code. The code in which the identification information of the patient is embedded may be, for example, a two-dimensional code, a one-dimensional code, or the like. The code may be positioned near a patient's bed.

Furthermore, the imaging control unit 111 can cause the imaging device 15 to image the field of view of the wearer via the transmission type display device 14 disposed in front of the eyes of the wearer. Furthermore, the imaging control unit 111 can cause the imaging device 15 to image the eyes of the wearer of the wearable terminal 10.

The image transmission unit 112 is a program that transmits the captured image generated by the imaging control unit 111 to the information processing apparatus 20. Specifically, the image transmission unit 112 transmits the code image to the information processing apparatus 20.

The display control unit 113 is a program for displaying various images on the display device 14. In the image displayed on the display device 14, information regarding the patient including biological information and the like are displayed.

The voice acquisition unit 114 is a program for acquiring the voice collected by the sound collection device 16. The voice information transmission unit 115 is a program that transmits voice information indicating the voice collected by the sound collection device 16.

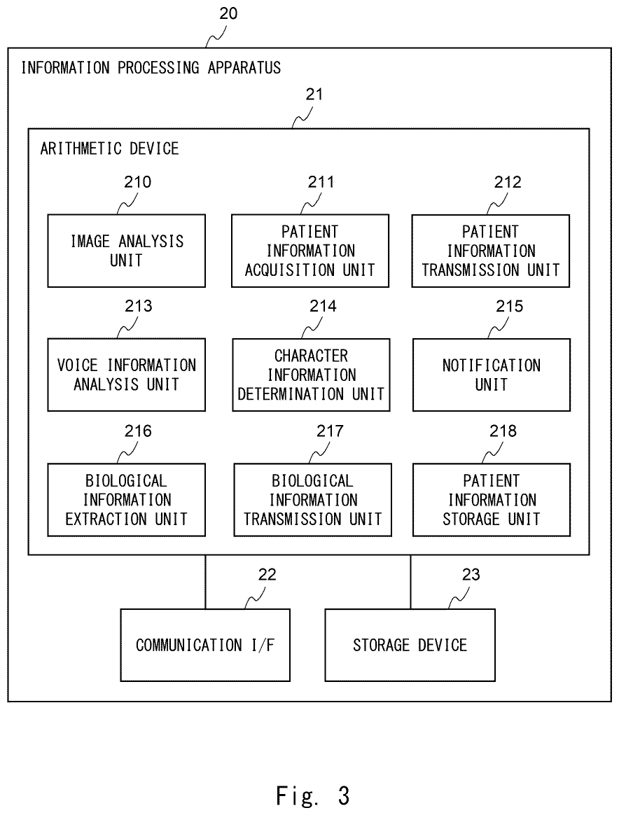

FIG. 3 is a schematic diagram illustrating a configuration of the information processing apparatus 20 in the present disclosure. The information processing apparatus 20 includes an arithmetic device 21, a communication interface 22, and a storage device 23.

The arithmetic device 21 is a device that performs overall control of the information processing apparatus 20. Specific examples of the arithmetic device 21 include a processor such as a CPU or a Graphics Processing Unit (GPU). The arithmetic device 21 executes a program stored in the storage device 23 to execute a method defined by the program.

The communication interface 22 is an interface that performs data communication between the information processing apparatus 20 and another device.

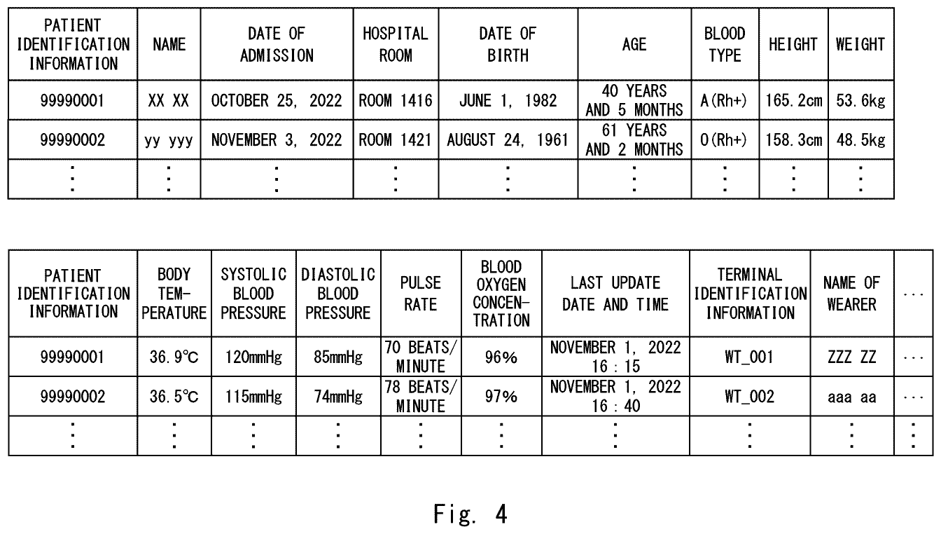

The storage device 23 is a storage device that stores various data such as a program executed by the arithmetic device 21 and data processed by the arithmetic device 21. The storage device 23 stores a data table (hereinafter, it is referred to as a “patient information table”.) in which patient information is registered. FIG. 4 is a diagram illustrating an example of a patient information table. In the patient information table, patient identification information, a name of the patient, a date of admission of the patient, a hospital room of the patient, a date of birth of the patient, an age of the patient, a height of the patient, a weight of the patient, a body temperature of the patient, a systolic blood pressure of the patient, a diastolic blood pressure of the patient, a pulse rate of the patient, a blood oxygen concentration of the patient, a last update date and time, a terminal identification information, a name of the wearer, and the like are registered. The last update date and time is the last date and time when the patient information has been updated. The terminal identification information is identification information of the wearable terminal 10 that has provided the voice including the biological information of the patient. In another example embodiment, the patient information table may be constructed in another storage device accessible by the information processing apparatus 20, for example, a data server or the like. Note that, in FIG. 4, the patient information table is divided for convenience of paper.

The arithmetic device 21 executes an image analysis unit 210, a patient information acquisition unit 211, a patient information transmission unit 212, a voice information analysis unit 213, a character information determination unit 214, a notification unit 215, a biological information extraction unit 216, a biological information transmission unit 217, and a patient information storage unit 218. These functional units can be realized by a program.

The image analysis unit 210 is a program that analyzes the code image. The image analysis unit 210 analyzes the code image and acquires the identification information of the patient embedded in the code image.

The patient information acquisition unit 211 is a program for acquiring the patient information from the patient information table. Specifically, the patient information acquisition unit 211 acquires patient information associated with the patient identification information acquired by the image analysis unit 210 from the patient information table.

The patient information transmission unit 212 is a program that transmits the patient information acquired from the patient information table by the patient information acquisition unit 211 to the wearable terminal 10.

The voice information analysis unit 213 is a program that analyzes voice information provided from the wearable terminal 10 and outputs character information included in the voice information. In the present example embodiment, a trained model capable of voice recognition can be adopted as the voice information analysis unit 213. The trained model can perform machine learning using training data. The training data include voice information including various conversations of people that used as input information and character information representing the conversation in characters that used as output information.

The character information determination unit 214 is a program that determines the character information output by the voice information analysis unit 213. More specifically, the character information determination unit 214 determines whether the character information indicating the recording start instruction of the biological information is included in the character information output by the voice information analysis unit 213. In a case where the instruction receiving unit 110 of the wearable terminal 10 receives the instruction to start the recording of the biological information of the patient, when the character information determination unit 214 determines that the character information indicating the recording start instruction of the biological information is included in the character information output from the voice information analysis unit 213, the information processing apparatus 20 shifts to the biological information recording mode. In the biological information recording mode, it is possible to extract, transmit, and save the biological information from the character information output by the voice information analysis unit 213.

In addition, the character information determination unit 214 determines whether the character information indicating the recording end instruction of the biological information is included in the character information output by the voice information analysis unit 213. After the information processing apparatus 20 shifts to the biological information recording mode, in a case where the character information determination unit 214 determines that the character information indicating the recording end instruction of the biological information is included in the character information output by the voice information analysis unit 213, the recording mode of the biological information ends, and the extraction, transmission, and saving of the biological information from the character information output by the voice information analysis unit 213 are stopped in the information processing apparatus 20.

The notification unit 215 is a program that transmits various notifications to the wearable terminal 10. The notification transmitted by the notification unit 215 includes a notification (hereinafter, it is referred to as “recording mode shift notification”.) that the information processing apparatus 20 has shifted to the biological information recording mode and a notification (hereinafter, it is referred to as “recording mode end notification”.) that the biological information recording mode of the information processing apparatus 20 has ended.

The biological information extraction unit 216 is a program for extracting biological information from the character information output by the voice information analysis unit 213. In the present example embodiment, a language model such as Large Language Models (LLM) can be adopted as the biological information extraction unit 216. The language model preferably specifies, as a prompt, a condition for outputting a character indicating the type of the biological information and a numerical value of the biological information. The character indicating the type of the biological information is, for example, a body temperature, a systolic blood pressure, a diastolic blood pressure, a heart rate, a blood oxygen concentration, and the like. The numerical value of the biological information is, for example, a value of the body temperature, a value of the systolic blood pressure, a value of the diastolic blood pressure, a value of the heart rate, a value of the blood oxygen concentration, and the like.

The biological information transmission unit 217 is a program that transmits the biological information extracted by the biological information extraction unit 216 to the wearable terminal 10.

The patient information storage unit 218 is a program for storing the patient information in the patient information table. For example, the patient information storage unit 218 stores the biological information extracted by the biological information extraction unit 216 in the patient information table in association with the identification information of the patient acquired by the image analysis unit 210.

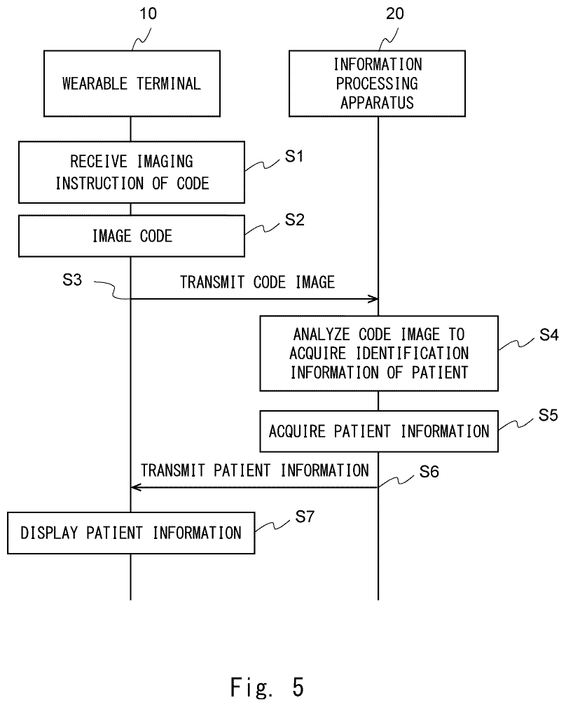

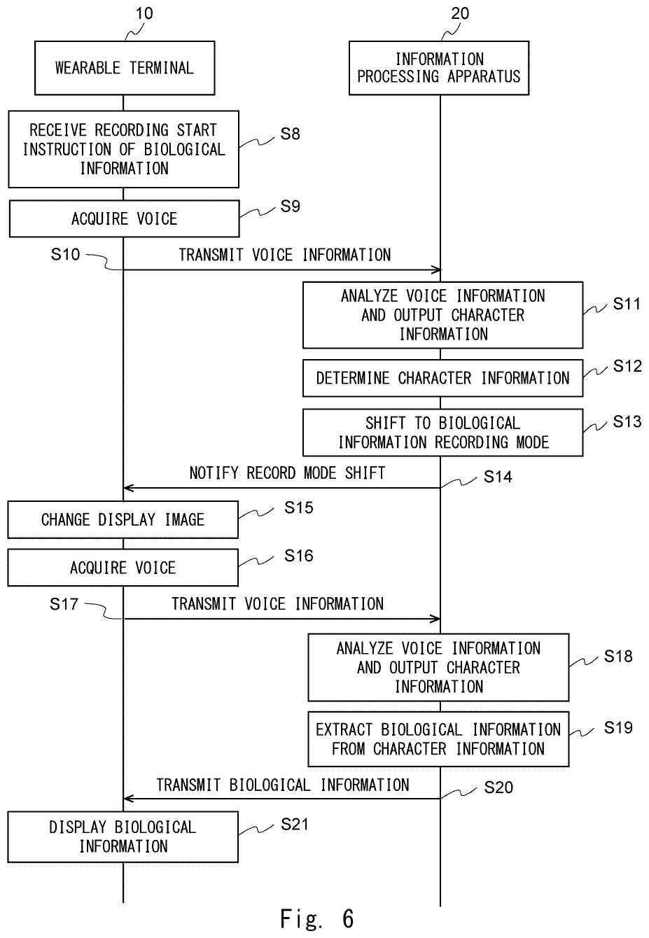

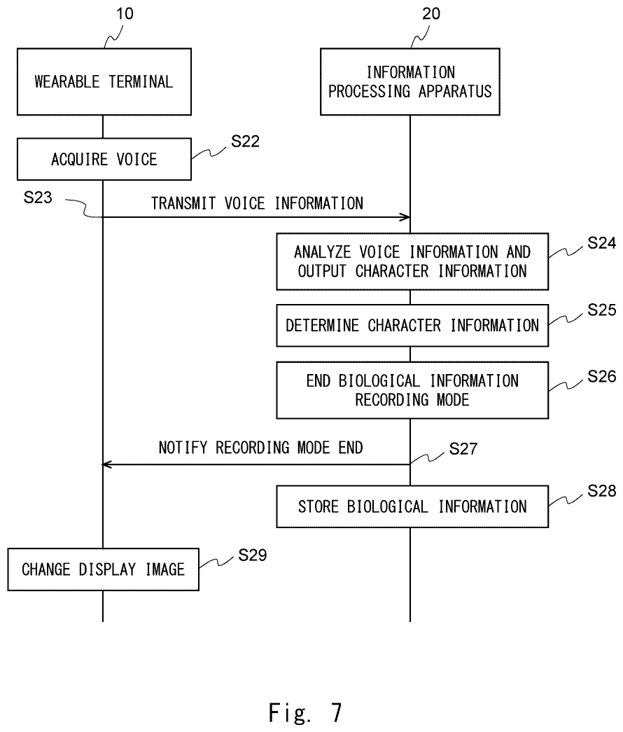

FIGS. 5 to 7 are sequence diagrams illustrating an example of processing executed in the biological information processing system 1. When the wearer of the wearable terminal 10 instructs the wearable terminal 10 to image the code in which the identification information of the patient is embedded, the instruction receiving unit 110 of the wearable terminal 10 receives the imaging instruction in Step S1.

When the instruction receiving unit 110 receives the imaging instruction, the imaging control unit 111 causes the imaging device 15 to image the code and generate the code image in Step S2. In Step S3, the image transmission unit 112 transmits the generated code image to the information processing apparatus 20 together with the identification information of the wearable terminal 10.

When the information processing apparatus 20 receives the code image transmitted by the wearable terminal 10, the image analysis unit 210 of the information processing apparatus 20 analyzes the code image to acquire the identification information of the patient in Step S4. In Step S5, the patient information acquisition unit 211 acquires the patient information associated with patient identification information from the patient information table. In Step S6, the patient information transmission unit 212 transmits the acquired patient information to the wearable terminal 10.

When the wearable terminal 10 receives the patient information transmitted by the information processing apparatus 20, the display control unit 113 of the wearable terminal 10 displays the received patient information on the display device 14 in Step S7. FIG. 8 is a diagram illustrating an example of the image displayed in Step S7. This image mainly displays the patient information registered in the patient information table.

Next, when the wearer of the wearable terminal 10 instructs the wearable terminal 10 to start recording the biological information of the patient, the instruction receiving unit 110 of the wearable terminal 10 receives the recording start instruction of the biological information of the patient in Step S8. When the instruction receiving unit 110 receives the recording start instruction of the biological information of the patient, the voice acquisition unit 114 acquires the voice collected by the sound collection device 16 in Step S9. In Step S10, the voice information transmission unit 115 transmits the voice information indicating the collected voice to the information processing apparatus 20.

When the information processing apparatus 20 receives the voice information transmitted by the wearable terminal 10, in Step S11, the voice information analysis unit 213 of the information processing apparatus 20 analyzes the received voice information and outputs the character information indicating a word included in the voice information. In Step S12, the character information determination unit 214 determines whether the character information indicating the recording start instruction of the biological information is included in the output character information. In a case where it is determined that the character information indicating the recording start instruction of the biological information is included in the output character information, the information processing apparatus 20 shifts to the biological information recording mode in Step S13. In Step S14, the notification unit 215 transmits the recording mode shift notification to the wearable terminal 10.

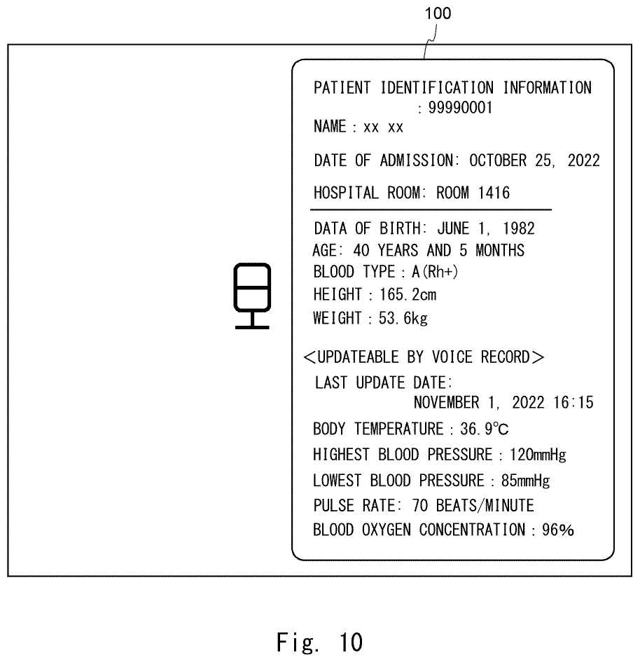

When the wearable terminal 10 receives the recording mode shift notification transmitted by the information processing apparatus 20, the display control unit 113 of the wearable terminal 10 changes the display image displayed on the display device 14 in Step S15. FIG. 10 is a diagram illustrating an example of the image displayed in Step S15. In this image, it is preferable to display that voice including biological information of the patient is recorded. For example, a character, a mark, or the like indicating that the voice is recorded can be adopted. At this time, the display control unit 113 can change a background color of an image 100 displayed in Step S7.

Next, in Step S16, the voice acquisition unit 114 acquires the voice collected by the sound collection device 16. In Step S17, the voice information transmission unit 115 transmits the voice information indicating the collected voice to the information processing apparatus 20.

When the information processing apparatus 20 receives the voice information transmitted by the wearable terminal 10, in Step S18, the voice information analysis unit 213 of the information processing apparatus 20 analyzes the received voice information and outputs character information indicating a word included in the voice information. In Step S19, the biological information extraction unit 216 extracts the biological information from the output character information.

In Step S20, the biological information transmission unit 217 transmits the extracted biological information to the wearable terminal 10. At this time, the biological information transmission unit 217 transmits characters indicating the type of biological information included in the extracted biological information in association with numerical values of the biological information. Specifically, the biological information transmission unit 217 transmits the character representing the body temperature and the numerical value of the body temperature in association with each other. In addition, the biological information transmission unit 217 transmits the character representing the systolic blood pressure and the numerical value of the systolic blood pressure in association with each other. Further, the biological information transmission unit 217 transmits the character representing the diastolic blood pressure and the numerical value of the diastolic blood pressure in association with each other. Further, the biological information transmission unit 217 transmits the character representing the blood oxygen concentration and the numerical value of the blood oxygen concentration in association with each other.

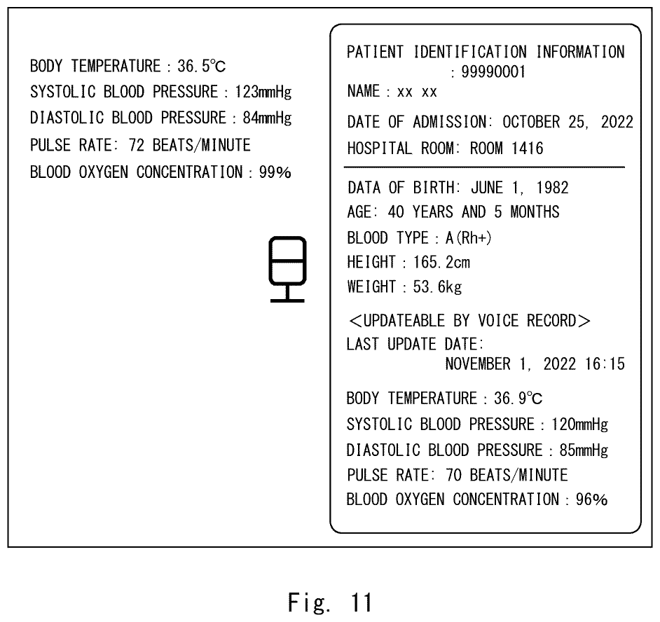

When the wearable terminal 10 receives the biological information transmitted by the information processing apparatus 20, the display control unit 113 of the wearable terminal 10 displays the received biological information on the display device 14 in Step S21. FIG. 11 is a diagram illustrating an example of the image displayed in Step S21. In this image, the character indicating the type of biological information included in the biological information transmitted by the information processing apparatus 20 and numerical values of the biological information are displayed in association with each other. In other words, the biological information of the patient extracted from the voice of the wearer of the wearable terminal 10 is displayed on the image.

Next, in Step S22, the voice acquisition unit 114 acquires the voice collected by the sound collection device 16. In Step S23, the voice information transmission unit 115 transmits the voice information indicating the collected voice to the information processing apparatus 20.

When the information processing apparatus 20 receives the voice information transmitted by the wearable terminal 10, in Step S24, the voice information analysis unit 213 of the information processing apparatus 20 analyzes the received voice information and outputs the character information indicating a word included in the voice information. In Step S25, the character information determination unit 214 determines whether the character information indicating the recording end instruction of the biological information is included in the output character information. In a case where it is determined that the character information indicating the recording end instruction of the biological information is included in the output character information, the information processing apparatus 20 ends the biological information recording mode in Step S26. In Step S27, the notification unit 215 transmits the recording mode end notification to the wearable terminal 10.

In Step S28, the patient information storage unit 218 stores the biological information extracted in Step S19 in the patient information table in association with the identification information of the patient acquired in Step S5. At this time, the patient information storage unit 218 stores the identification information of the wearable terminal 10 received from the wearable terminal 10 and the last update date and time corresponding to the date and time when the biological information is stored in the patient information table in association with the identification information of the patient. Further, the patient information storage unit 218 registers the name of the wearer of the wearable terminal 10 in association with the identification information of the wearable terminal 10. The patient information storage unit 218 refers to another data table in which the identification information of the wearable terminal 10 and the name of the wearer are registered in association with each other, and can specify the name of the wearer registered in the patient information table.

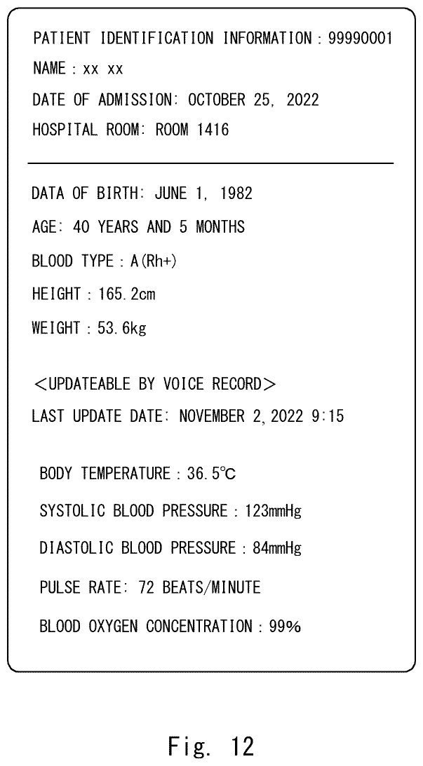

When the wearable terminal 10 receives the recording mode end notification transmitted by the information processing apparatus 20, the display control unit 113 of the wearable terminal 10 changes the display image displayed on the display device 14 in Step S29. FIG. 12 is a diagram illustrating an example of the image displayed in Step S29. The updated biological information is displayed on this image.

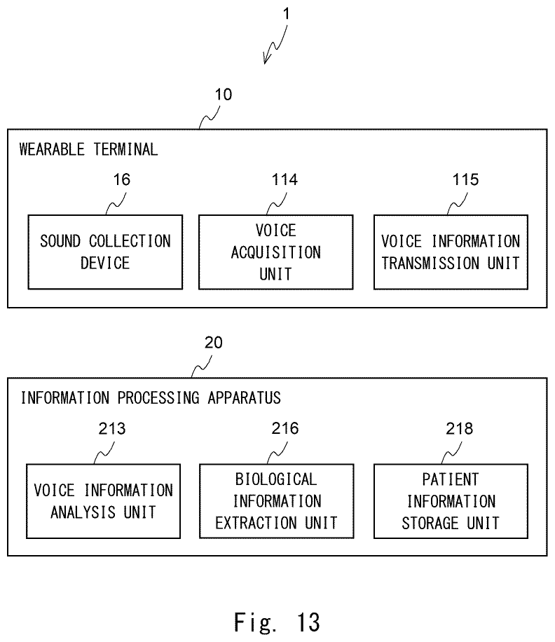

FIG. 13 is a diagram illustrating main components included in the biological information processing system 1. The biological information processing system 1 includes the wearable terminal 10 worn by the medical worker and acquiring the voice, and the information processing apparatus 20 that extracts and stores the biological information of the patient from the voice acquired by the wearable terminal 10. The wearable terminal 10 includes the sound collection device 16 that collects the voice, the voice acquisition unit 114 that acquires the voice collected by the sound collection device 16, and the voice information transmission unit 115 that transmits voice information indicating the sound acquired by the voice acquisition unit 114 to the information processing apparatus 20. The information processing apparatus 20 includes the voice information analysis unit 213 that analyzes the voice information transmitted by the voice information transmission unit 115 and outputs the character information indicating the word included in the voice information, the biological information extraction unit 216 that extracts the biological information of the patient from the character information output by the voice information analysis unit 213, and the patient information storage unit 218 that stores the biological information of the patient extracted by the biological information extraction unit 216 in the storage device 23.

By adopting this configuration, the medical worker worn by the wearable terminal 10 can move to a place where the patient is and record the measurement result of the biological information of the patient using voice. Therefore, convenience in recording the measurement result of the biological information of the patient can be improved, and the burden on the medical worker can be reduced.

Furthermore, in the above-described example embodiment, the biological information extraction unit 216 may be a language model. When the character information is input into the language model as the input information, the language model outputs the biological information included in the character information as the output information. In this language model, a condition for outputting a character indicating the type of biological information and a numerical value of the biological information is specified. As a result, the biological information extraction unit 216 can appropriately output the character indicating the type of biological information and the numerical value of the biological information.

Furthermore, the wearable terminal 10 can include the transmission type display device 14 disposed in front of the eyes of the wearer of the wearable terminal 10, the imaging device 15 capable of imaging the field of view of the wearer via the display device 14, and the instruction receiving unit 110 that receives the instruction from the wearer. The instruction receiving unit 110 determines whether the position of the fingertip of the wearer in the captured image generated by the imaging device 15 is in the predetermined area of the captured image corresponding to the predetermined area of the image displayed on the display device 14. In a case where it is determined that the position of the fingertip of the wearer is in the predetermined area of the captured image, the instruction receiving unit 110 receives the instruction to start the recording of the biological information of the patient.

As a result, the wearer can instruct to start the recording of the biological information of the patient when the fingertip of the wearer as viewed from the wearer is in the predetermined area of the image displayed on the display device 14. Therefore, it is possible to improve convenience when recording the measurement result of the biological information of the patient.

Furthermore, the wearable terminal 10 may include the imaging device capable of imaging the field of view of the wearer via the display device 14. The instruction receiving unit 110 determines whether the position of the center point of the iris of the wearer in the captured image generated by the imaging device 15 is in the predetermined area of the captured image corresponding to the predetermined area of the image displayed on the display device 14. In a case where it is determined that the position of the center point of the iris of the wearer is in the predetermined area of the captured image, the instruction receiving unit 110 receives the instruction to start the recording of the biological information of the patient.

As a result, the wearer can give the instruction to start the recording of the biological information of the patient by moving the line of sight such that the position of the center point of the iris is in the predetermined area of the captured image corresponding to the predetermined area of the image displayed on the display device 14. Therefore, it is possible to improve convenience when recording the measurement result of the biological information of the patient.

In the above example, a program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

While the present disclosure has been particularly shown and described with reference to example embodiments thereof, the present disclosure is not limited to these example embodiments. It will be understood by those of ordinary skill in the art that various changes in form and details may be made therein without departing from the sprit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

Some or all of the above-described example embodiments may be described as in the following Supplementary Notes, but are not limited to the following Supplementary Notes.

### (Supplementary Note 1)

A biological information processing system including:
a wearable terminal worn by a medical worker and configured to acquire a voice; and
an information processing apparatus configured to extract and store biological information of a patient from the voice acquired by the wearable terminal, in which
the wearable terminal includes:
   a sound collection unit configured to collect the voice;
   a voice acquisition unit configured to acquire the voice collected by the sound collection unit; and
   a voice information transmission unit configured to transmit voice information indicating the voice acquired by the voice acquisition unit to the information processing apparatus, and
the information processing apparatus includes:
   a voice information analysis unit configured to analyze the voice information transmitted by the voice information transmission unit and output character information indicating a word included in the voice information;
   a biological information extraction unit configured to extract biological information of the patient from the character information output by the voice information analysis unit; and
   a patient information storage unit configured to store the biological information of the patient extracted by the biological information extraction unit in a storage device.

### (Supplementary Note 2)

The biological information processing system according to Supplementary Note 1, in which
the biological information extraction unit is a language model that uses the character information as input information and biological information included in the character information as output information, and
in the language model, a condition for outputting a character indicating a type of the biological information and a numerical value of the biological information is designated.

### (Supplementary Note 3)

The biological information processing system according to Supplementary Note 1 or 2, in which the biological information includes at least one of a body temperature, a blood pressure, a heart rate, and a blood oxygen concentration of the patient.

### (Supplementary Note 4)

The biological information processing system according to any one of Supplementary Notes 1 to 3, in which
the wearable terminal includes:
a transmissive display unit disposed in front of eyes of a wearer of the wearable terminal;
an imaging device capable of imaging a field of view of the wearer via the display unit; and
an instruction receiving unit configured to receive an instruction from the wearer, and wherein
the instruction receiving unit determines whether a position of a fingertip of the wearer in a captured image generated by the imaging device is in a predetermined area of the captured image corresponding to a predetermined area of an image displayed on the display unit, and
the instruction receiving unit receives an instruction to start recording of the biological information of the patient in a case where it is determined that the position of the fingertip of the wearer is in the predetermined area of the captured image.

### (Supplementary Note 5)

The biological information processing system according to Supplementary Note 4, in which the biological information extraction unit starts extraction of the biological information of the patient from the character information when the character information output by the voice information analysis unit includes a character instructing to start recording of the biological information of the patient in a case where the instruction receiving unit receives an instruction to start recording of the biological information of the patient.

### (Supplementary Note 6)

The biological information processing system according to any one of Supplementary Notes 1 to 3, in which
the wearable terminal includes:
a transmissive display unit disposed in front of eyes of a wearer of the wearable terminal;
an imaging device capable of imaging an eye of the wearer; and
an instruction receiving unit that receives an instruction from the wearer, and wherein
the instruction receiving unit determines whether a position of a center point of an iris of the wearer in a captured image generated by the imaging device is in a predetermined area of the captured image corresponding to a predetermined area of an image displayed on the display unit, and
the instruction receiving unit receives an instruction to start recording of the biological information of the patient in a case where it is determined that the position of the center point of the iris of the wearer is in a predetermined area of the captured image.

### (Supplementary Note 7)

The biological information processing system according to Supplementary Note 6, in which the biological information extraction unit starts extraction of the biological information of the patient from the character information when the character information output by the voice information analysis unit includes a character instructing to start recording of the biological information of the patient in a case where the instruction receiving unit receives an instruction to start recording of the biological information of the patient.

### (Supplementary Note 8)

A biological information processing method, causing a computer to:
analyze voice information indicating a voice acquired by a wearable terminal worn by a medical worker and output character information indicating a word included in the voice information;
extract the biological information of the patient from the output character information; and
store the extracted biological information of the patient in a storage device.

### (Supplementary Note 9)

The biological information processing method according to Supplementary Note 8, in which
the computer extracts the biological information of the patient from the output character information by using a language model that uses the character information as input information and biological information included in the character information as output information, and
in the language model, a condition for outputting a character indicating a type of the biological information and a numerical value of the biological information is designated.

### (Supplementary Note 10)

The biological information prescription method according to Supplementary Note 8 or 9, in which the biological information includes at least one of a body temperature, a blood pressure, a heart rate, and a blood oxygen concentration of the patient.

### (Supplementary Note 11)

The biological information processing method according to any one of Supplementary Notes 8 to 10, in which in a case where an instruction to start recording the biological information of the patient is received, the computer starts extracting the biological information of the patient from the character information when the output character information includes a character instructing to start recording of the biological information of the patient.

### (Supplementary Note 12)

A non-transitory recording medium storing a biological information processing program, causing a computer to:
analyze voice information indicating a voice acquired by a wearable terminal worn by a medical worker and output character information indicating a word included in the voice information;
extract the biological information of the patient from the output character information; and
store the extracted biological information of the patient in a storage device.

### (Supplementary Note 13)

The non-transitory recording medium according to Supplementary Note 12, further causing the computer to extract the biological information of the patient from the output character information by using a language model that uses the character information as input information and biological information included in the character information as output information,
in which in the language model, a condition for outputting a character indicating a type of the biological information and a numerical value of the biological information is designated.

### (Supplementary Note 14)

The non-transitory recording medium according to Supplementary Note 12 or 13, in which the biological information includes at least one of a body temperature, a blood pressure, a heart rate, and a blood oxygen concentration of the patient.

### (Supplementary Note 15)

The non-transitory recording medium according to any one of Supplementary Notes 12 to 14, in which in a case where an instruction to start recording the biological information of the patient is received, the computer is caused to start extracting the biological information of the patient from the character information when the output character information includes a character instructing to start recording of the biological information of the patient.

### (Supplementary Note 16)

An information processing apparatus including:
a voice information analysis unit configured to analyze voice information indicating a voice acquired by a wearable terminal worn by a medical worker and output character information indicating a word included in the voice information;
a biological information extraction unit configured to extract biological information of a patient from character information output by the voice information analysis unit; and
a patient information storage unit configured to store the biological information of the patient extracted by the biological information extraction unit in a storage device.

### (Supplementary Note 17)

The information processing apparatus according to Supplementary Note 16, in which
the biological information extraction unit is a language model that uses the character information as input information and biological information included in the character information as output information, and
in the language model, a condition for outputting a character indicating a type of the biological information and a numerical value of the biological information is designated.

### (Supplementary Note 18)

The information processing apparatus according to Supplementary Note 16 or 17, in which the biological information includes at least one of a body temperature, a blood pressure, a heart rate, and a blood oxygen concentration of the patient.

### (Supplementary Note 19)

The information processing apparatus according to any one of

Supplementary Notes 16 to 18, in which the biological information extraction unit starts extraction of the biological information of the patient from the character information when the character information output by the voice information analysis unit includes a character instructing to start recording of the biological information of the patient in a case where the instruction receiving unit receives an instruction to start recording of the biological information of the patient.

Some or all of the elements described in any supplementary notes may be applied to various hardware, software, recording means for recording software, systems, and methods.

What is claimed is:

1. A biological information processing system comprising:
a wearable terminal worn by a medical worker and configured to acquire a voice; and
an information processing apparatus configured to extract and store biological information of a patient from the voice acquired by the wearable terminal, wherein
the wearable terminal includes:
a sound collection unit configured to collect the voice;
a voice acquisition unit configured to acquire the voice collected by the sound collection unit; and
a voice information transmission unit configured to transmit voice information indicating the voice acquired by the voice acquisition unit to the information processing apparatus, and
the information processing apparatus includes:
a voice information analysis unit configured to analyze the voice information transmitted by the voice information transmission unit and output character information indicating a word included in the voice information;
a biological information extraction unit configured to extract biological information of the patient from the character information output by the voice information analysis unit; and
a patient information storage unit configured to store the biological information of the patient extracted by the biological information extraction unit in a storage device.

2. The biological information processing system according to claim 1, wherein
the biological information extraction unit is a language model that uses the character information as input information and biological information included in the character information as output information, and
in the language model, a condition for outputting a character indicating a type of the biological information and a numerical value of the biological information is designated.

3. The biological information processing system according to claim 1, wherein the biological information includes at least one of a body temperature, a blood pressure, a heart rate, and a blood oxygen concentration of the patient.

4. The biological information processing system according to claim 1, wherein
the wearable terminal includes:
a transmissive display unit disposed in front of eyes of a wearer of the wearable terminal;
an imaging device capable of imaging a field of view of the wearer via the display unit; and
an instruction receiving unit configured to receive an instruction from the wearer, wherein
the instruction receiving unit determines whether a position of a fingertip of the wearer in a captured image generated by the imaging device is in a predetermined area of the captured image corresponding to a predetermined area of an image displayed on the display unit, and
the instruction receiving unit receives an instruction to start recording of the biological information of the patient in a case where it is determined that the position of the fingertip of the wearer is in the predetermined area of the captured image.

5. The biological information processing system according to claim 4, wherein the biological information extraction unit starts extraction of the biological information of the patient from the character information when the character information output by the voice information analysis unit includes a character instructing to start recording of the biological information of the patient in a case where the instruction receiving unit receives an instruction to start recording of the biological information of the patient.

6. The biological information processing system according to claim 1, wherein
the wearable terminal includes:
a transmissive display unit disposed in front of eyes of a wearer of the wearable terminal;
an imaging device capable of imaging an eye of the wearer; and
an instruction receiving unit that receives an instruction from the wearer, and wherein
the instruction receiving unit determines whether a position of a center point of an iris of the wearer in a captured image generated by the imaging device is in a predetermined area of the captured image corresponding to a predetermined area of an image displayed on the display unit, and
the instruction receiving unit receives an instruction to start recording of the biological information of the patient in a case where it is determined that the position of the center point of the iris of the wearer is in a predetermined area of the captured image.

7. The biological information processing system according to claim 6, wherein the biological information extraction unit starts extraction of the biological information of the patient from the character information when the character information output by the voice information analysis unit includes a character instructing to start recording of the biological information of the patient in a case where the instruction receiving unit receives an instruction to start recording of the biological information of the patient.

8. A biological information processing method, causing a computer to:
analyze voice information indicating a voice acquired by a wearable terminal worn by a medical worker and output character information indicating a word included in the voice information;
extract the biological information of the patient from the output character information; and
store the extracted biological information of the patient in a storage device.

9. The biological information processing method according to claim 8, wherein
the computer extracts the biological information of the patient from the output character information by using a language model that uses the character information as input information and biological information included in the character information as output information, and
in the language model, a condition for outputting a character indicating a type of the biological information and a numerical value of the biological information is designated.

10. The biological information processing method according to claim 8, wherein the biological information includes at least one of a body temperature, a blood pressure, a heart rate, and a blood oxygen concentration of the patient.

11. The biological information processing method according to claim 8, wherein in a case where an instruction to start recording the biological information of the patient is received, the computer starts extracting the biological information of the patient from the character information when the output character information includes a character instructing to start recording of the biological information of the patient.

12. A non-transitory recording medium storing a biological information processing program, causing a computer to:
analyze voice information indicating a voice acquired by a wearable terminal worn by a medical worker and output character information indicating a word included in the voice information;
extract the biological information of the patient from the output character information; and
store the extracted biological information of the patient in a storage device.

13. The non-transitory recording medium according to claim 12, further causing the computer to extract the biological information of the patient from the output character information by using a language model that uses the character information as input information and biological information included in the character information as output information,
wherein in the language model, a condition for outputting a character indicating a type of the biological information and a numerical value of the biological information is designated.

14. The non-transitory recording medium according to claim 12, wherein the biological information includes at least one of a body temperature, a blood pressure, a heart rate, and a blood oxygen concentration of the patient.

15. The non-transitory recording medium according to claim 12, wherein in a case where an instruction to start recording the biological information of the patient is received, the computer is caused to start extracting the biological information of the patient from the character information when the output character information includes a character instructing to start recording of the biological information of the patient.
